(19) United States
(12) Patent Application Publication
(10) Pub. No.: US 2022/0317069 A1
(43) Pub. Date: Oct. 6, 2022

(54) METHOD AND SYSTEM FOR CLASSIFICATION OF SAMPLES

(71) Applicant: SECURITY MATTERS LTD., D.N. Hevel Eilot (IL)

(72) Inventors: Yair GROF, Rehovot (IL); Dmitrijs DOCENKO, Jerusalem (IL); Mirit KAGARLITSKY, Ramat Gan (IL); Nataly TAL, Nes Ziona (IL); Nadav YORAN, Tel Aviv (IL); Haggai ALON, Kibbutz Naan (IL)

(73) Assignee: SECURITY MATTERS LTD., D.N. Hevel Eilot (IL)

(21) Appl. No.: 17/594,406

(22) PCT Filed: Apr. 5, 2020
(86) PCT No.: PCT/IL2020/050409

§ 371 Date: Oct. 14, 2021

Related U.S. Application Data
(60) Provisional application No. 62/833,956, filed on Apr. 15, 2019.

(51) Int. Cl. G01N 23/207; G01N 23/223; G01N 33/38

(52) U.S. Cl. CPC G01N 23/2076; G01N 23/223; G01N 33/381; G01N 2223/076; G01N 2223/616; G01N 2223/304

(57) ABSTRACT

A method and system are provided for model-based analysis of samples of interest and management of sample classification. Predetermined modeled data is provided including data indicative of K models for respective K measurement schemes based on a predetermined function having a spectral line shape, data indicative of M characteristic vectors of M predetermined group to which different samples relate, and data indicative of a common vector of weights for the M groups. A data processor utilizes the data and operates to apply model-based processing to measured spectral data of a sample of interest using the predetermined modeled data, and generate classification data indicative of relation of the specific sample of interest to one of the M predetermined groups.

### TECHNOLOGICAL FIELD

The present invention is in the field of modeling and model-based analysis of measurements of samples and classifying the samples.

### GENERAL DESCRIPTION

The inventors have found that in various industries, in particular those dealing with manufacture and distribution of such objects as minerals, precious stones, such as diamonds, there might be a need to identify/classify an object/sample of interest as relating to a specific group of objects/samples having common or similar characteristics. These may include one or more structural parameters of an area of object's origination, and/or a geographical location of an area of object's origination.

The inventors have also found that objects relating to the same group (i.e. group having predefined group-related or group-unique characteristics) can be classified, in a manner distinguishing them from one or more other groups, by the objects' spectra. For example, such spectral data may be indicative of X-ray Fluorescence (XRF) response of the object/sample to X-ray or Gamma-ray radiation.

The technique of the present invention thus provides a novel modeling technique enabling creation of novel model data to be used in classifying a sample of interest to a related group based on measured spectral data. In other words, the present invention provides certain novel model-based approach for associating a sample of interest, based on its measured spectrum/spectra to one of multiple predefined groups.

Thus, according to one broad aspect of the invention, there is provided a method for model-based analysis of samples of interest, comprising analysis of certain reference spectral data relating to reference samples relating to the two or more different groups having predetermined different characteristics, and creation of the modeled data. More specifically, the method comprises:

providing reference data indicative of spectral measurements of a number K of measurement schemes performed on a plurality of N reference samples relating to M groups, which have predetermined different characteristics, the reference data comprising raw measured data including a plurality of (N×K) measured reference spectra, and comprising data indicative of correspondence of each of the reference samples to a respective one of said M groups;

processing said plurality of the (N×K) measured reference spectra to determine K models corresponding to said K measurement schemes, respectively, the models being based on a predetermined function having a spectral line shape, and relating to the respective measurement scheme;

fitting each of said K models with each of the N measured reference spectra corresponding to the respective measurement scheme, and creating, for each of the reference samples, a vector representation of the sample's reference spectra for said number K of measurement schemes, thereby representing each of the reference samples by the respective vector of components;

utilizing said data indicative of the correspondence of each of the samples to the respective one of said M groups, and, for each group, analyzing the vectors of components of the samples relating to the group, and determining data indicative of a characteristic vector of the group; and

determining weight parameters of a distance function that maximizes a combined likelihood for associating all the vectors of components of the reference samples with their respective groups, based on the distance function between the vector of components of the reference sample and the characteristic vector of the group, thereby providing a common vector of said weight parameters of the distance function;

storing modeled data comprising data indicative of the K models for the respective K measurement schemes, data indicative of the characteristic vectors of the group, and data indicative of the common vector of weights for the M groups, thereby enabling to classify a sample of interest to relate it to one of said M groups, by model-based analysis of raw measured spectral data of the sample of interest using said modeled data.

Classification of a sample on interest (a so-called “unknown sample”) from raw measured spectral data of said sample performed using one or more measurement schemes, can be done as follows:

based on the raw measured spectral data of the sample of interest, K data pieces are determined corresponding to K measured spectra of the sample of interest under the K measurement schemes, respectively,

the model-based analysis are applied to the K data pieces, including:
using the stored K models and fitting each of said K measured spectra to the sample of interest to the respective one of the stored K models, and, based on best fit conditions for each of the K measured spectra, creating a combined vector representation of the sample of all of said K measurement schemes;
applying said distance function with said common vector of weights to determine distances of said combined vector representation of the sample to each of the characteristic vectors of the groups, and associating said sample with group for which the determined distance is minimal.

Generally, the technique of the present invention provides for creation of the proper modeled data, as well as provides for properly classifying the unknown sample. Preferably, however, two or more different measurement schemes are used. The measurement schemes/conditions may differ from one another in one or more parameters. In some embodiments, such parameters may include one or more of the following: the primary radiation intensity, distribution of the energy of photons in the primary radiation, (which can be set by electric current and voltage of a tube emitting the primary radiation, and/or filters at the radiation emitting source. Additionally, or alternatively, variation of one or more of the following can be used in different measurement schemes: collimation of the primary radiation signal, a size of the irradiation spot, filtering of the radiation response signal at the detector, geometrical configuration of the radiation source, relative orientation and accommodation of the surface of the sample and the radiation source and/or radiation detector (e.g. the angles and distances between the surface of the sample, the radiation source, and the detector) may affect the measurement of the spectrums and may be varied to create different measurement conditions. Furthermore, for some or all spectra being measured, the sample may be rotated around one or more axes so that the counts of emitted radiation portions from the sample during various sample orientations are collected in a single spectrum.

The model, created per measurement scheme, is configured as a mixture model, being based on the predetermined function of the spectral line shape, and a certain piecewise (or hybrid) function being piecewise linear or piecewise polynomial function. Such function having the spectral line shape may include Lorentzian, Gaussian and/or Voigt functions.

The group' characteristic vector includes average values of the components in the vectors of components representing the reference samples of the same group. The distance function is associated with the average values and standard deviation, thereby describing amount of spread of the values of the components in the vectors of components.

The processing of the plurality of the (N×K) measured reference spectra to determine the K models may be performed as follows:

for each i-th plurality of the measured reference spectra of the N reference samples corresponding to the i-th measurement scheme (i=1, . . . , K), an average measured reference spectrum is determined; and

a predetermined transformation is applied to each i-th average measured reference spectrum according to the predetermined function having the spectral line shape, to obtain a respective i-th model corresponding to the i-th measurement scheme, thereby obtaining the K models for the K measurement schemes.

According to another broad aspect of the invention, it provides a data analysis system for modeling measurements on samples. The system includes a measurement system (e.g. for measuring X-ray Fluorescence (XRF) response of the sample to X-ray or Gamma-ray radiation), and a control configured and operable to determine, based on the measured reference data, modeled data enabling further classification of a sample of interest. More specifically, the measurement system is configured and operable to perform spectral measurements on a plurality of N reference samples relating to M groups of predetermined different characteristics, under a number K of measurement schemes, and generate measured reference data including a plurality of (N×K) measured reference spectra in association with said M groups. The control system includes:

a model creation module configured and operable to process said plurality of the (N×K) measured reference spectra and determine K models corresponding to said K measurement schemes, respectively, the models being based on a predetermined function having a spectral line shape, and relating to the respective measurement scheme;

a fitting module configured and operable to carry out the following: for each of said K models, fitting the model with each of the N measured reference spectra corresponding to the respective measurement scheme; and creating, for each of the reference samples, a vector representation of the sample's reference spectra for said number K of measurement schemes, thereby representing each of the reference samples by the respective vector of components;

a group characterization module configured and operable to utilize data indicative of correspondence of each of the reference samples to the respective one of said M groups, and analyze, for each group, the vectors of components of the samples relating to the group, and determining data indicative of a characteristic vector of the group;

a weighting module configured and operable to determine weight parameters of a distance function that maximizes a combined likelihood for associating all the vectors of components of the reference samples with their respective groups, based on the distance function between the vector of components of the reference sample and the characteristic vector of the group, thereby providing a common vector of said weight parameters of the distance function; and

an output utility configured and operable to generate the modeled data to be stored, said modeled data comprising: data indicative of the K models for the respective K measurement schemes, data indicative of the characteristic vectors of the group, and data indicative of the common vector of weights for the M groups.

The invention, in its yet further broad aspect, provides a sample classification system comprising:

a measurement system configured and operable to perform spectral measurements on samples under a number K of measurement schemes, and generate, for each of the measured samples, measured spectral data comprising K measured data pieces indicative of measured spectra corresponding to the K measurement schemes, respectively;

a control system configured and operable to communicate with the measurement system to receive the measured spectral data of a sample of interest, and configured and operable to communicate with a memory storing predetermined modeled data comprising data indicative of K models for the respective K measurement schemes based on a predetermined function having a spectral line shape, data indicative of M characteristic vectors of M predetermined group to which different samples relate, and data indicative of a common vector of weights for the M groups, said control system comprising a data processor configured and operable to apply model-based processing to the received measured spectral data of the sample of interest using said predetermined modeled data, and generate classification data indicative of relation of said specific sample of interest to one of said M predetermined groups.

In some embodiments, the control system comprises:

a fitting module configured and operable to carry out the following: for each of said K measured spectra, fitting the measured spectrum to the respective model, and obtaining K best fit condition spectra; and using said K best fit condition spectra to create a combined vector representation of the sample of interest for all said K measurement schemes;

a classifier module configured and operable to utilize a predetermined distance function with said common vector of weights and determine a distances of said combined vector representation of the sample of interest to each of said M characteristic vectors of the M groups, and associate said sample of interest with a group for which the determined distance is minimal.

In some embodiments, the control system is further configured and operable to determine the predetermined modeled data, based on the measured spectral data corresponding to spectral reference measurements for the number K of said measurement schemes performed on a plurality of N reference samples relating to said M groups, where the spectral reference data comprises a plurality of (N×K) measured reference spectra, and comprises data indicative of correspondence of each of the reference samples to a respective one of said M groups. The control system comprises:

a model creation module configured and operable to process said plurality of the (N×K) measured reference spectra and determine the K models corresponding to said K measurement schemes;

a fitting module configured and operable to carry out the following: for each of said K models, fitting the model with each of the N measured reference spectra corresponding to the respective measurement scheme; and creating, for each of the reference samples, a vector representation of the sample's reference spectra for said number K of measurement schemes, thereby representing each of the reference samples by the respective vector of components;

a group characterization module configured and operable to utilize data indicative of correspondence of each of the reference samples to the respective one of said M groups, and analyze, for each group, the vectors of components of the samples relating to the group, and determining data indicative of the characteristic vector of the group; and

a weighting module configured and operable to determine weight parameters of the predetermined distance function that maximizes a combined likelihood for associating all the vectors of components of the reference samples with their respective groups, based on the distance function between the vector of components of the reference sample and the characteristic vector of the group, thereby providing said common vector of said weight parameters of the distance function.

According to yet further broad aspect of the invention, it provides a control system for use in managing sample classification. The control system is configured and operable to communicate with a measured data provider to receive measured spectral data of a sample of interest, and is configured and operable to communicate with a memory storing predetermined modeled data comprising data indicative of K models for respective K measurement schemes based on a predetermined function having a spectral line shape, data indicative of M characteristic vectors of M predetermined group to which different samples relate, and data indicative of a common vector of weights for the M groups. The control system comprises a data processor configured and operable to apply model-based processing to the received measured spectral data of the sample of interest using said predetermined modeled data, and generate classification data indicative of relation of said specific sample of interest to one of said M predetermined groups.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 is a block diagram of a data analysis system of the present invention for creation of modeled data for classifying samples;

FIG. 2A is a block diagram exemplifying reference spectral data used for creation of the modeled data;

FIG. 2B is a block diagram exemplifying sample's spectral data transformed into vector of components representation of the sample;

FIG. 3 is a flow diagram exemplifying a method of the invention for using the reference spectral data and creation of the modeled data;

FIG. 4 is a flow diagram of a method of the present invention for classifying a sample by model-based processing of raw measured spectral data of the sample, using the modeled data created by the method of the invention; and

FIG. 5 is a flow diagram of the main steps in a method of the invention for clustering unclassified samples.

### DETAILED DESCRIPTION OF EMBODIMENTS

The present invention provides a novel approach for classifying a sample, based on sample's measured spectra, as relating to/associated with a characteristic group of similar/related samples. As described above, samples/objects of some types, such as minerals, precision stones (in particular diamonds) need to be identified by their association/relation to a specific group. The group may be descriptive by one or more structural parameters of an area of sample's origination, and/or a geographical location of an area of sample's origination. Samples relating to the same group (i.e. group having predefined group-related and group-unique characteristics) can be classified based on their spectral data, in a manner distinguishing them from samples/spectra of one or more other groups.

The present invention provides a novel technique for creation of novel modeled data to be used for classifying a sample of interest to a related group based on raw measured spectral data of the sample.

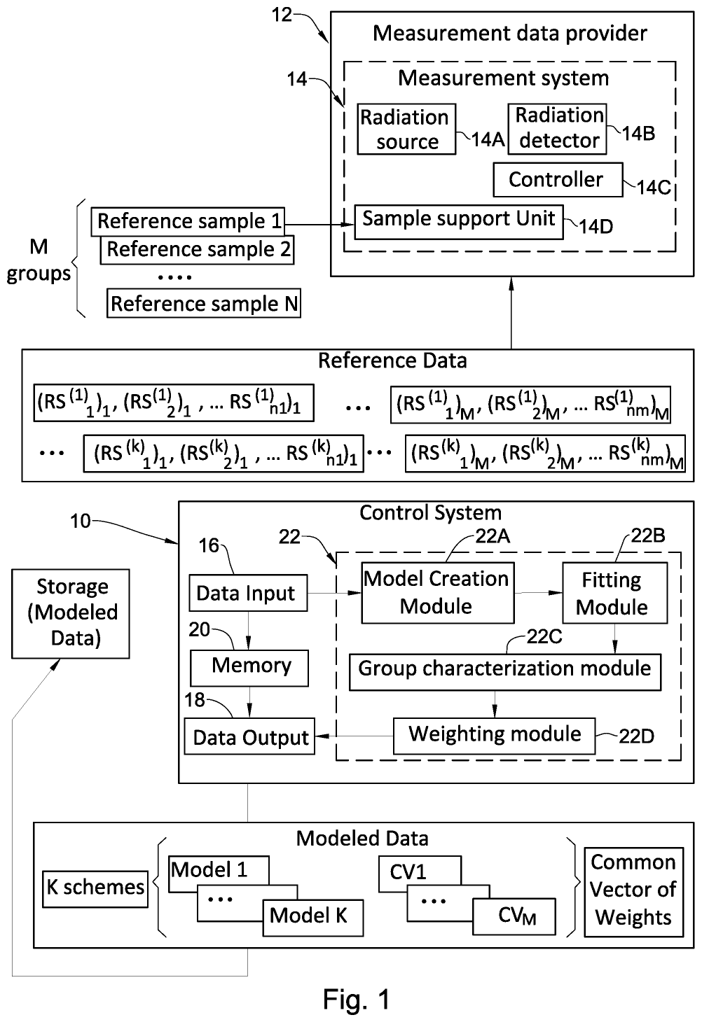

Reference is made to FIG. 1, illustrating, by way of a block diagram, a data analysis system 10 of the present invention for creation of modeled data to be further used for classifying samples. The system 10 is a control system configured for data communication with a measured data provider 12. The control system 10 is typically a computer system, and may be part of/integral with the measured data provider, or may communicate with the measured data provider via a communication network, using any 10 known suitable communication technique and data protocol, e.g. using cloud computing technique. The construction and operation of data communication networks and protocols between remote entities are well known per se, and do not form part of the present invention, and therefor need not be described in details.

The measured data provider may be constituted by a measurement system itself 14, as shown in the present not limiting example, or may be a separate storage device in data communication with the measurement system, using any known suitable communication techniques. As shown in this specific example, the measurement system 14 includes a radiation source 14A, a radiation detector 14B, a controller 14C, as well as a sample support unit 14D.

It should be understood, although not specifically shown, that the measurement system may also include various other units and hardware/software utilities for managing the measurement procedures, which do not form part of the present invention, and therefore need not be specifically described, except to note the following: For the purposes of the present invention, measured data needed for creation of modelled data includes, for each sample, a predetermined number K (K≥1) measured spectra obtained under different measurement conditions/schemes. Generally, measurements using a single measurement scheme (K=1) might be enough for the modeled data creation. However, when dealing with spectral measurements, and moreover volumetric samples of various shapes and geometries, provision of multiple spectra corresponding to different measurement schemes is preferred.

In some embodiments, suitable for measurements on precision stones, in particular diamonds, which might have various markings on their surfaces and/or within the volume, spectral data may be indicative of X-ray Fluorescence (XRF) response of the sample to X-ray or Gamma-ray radiation. Accordingly, the radiation source 14A may be X-ray or Gamma-ray radiation source configured to irradiate the sample by primary exciting radiation to induce emission of secondary X-ray Fluorescence (XRF) response from the sample, and the radiation detector 14B is configured for detection of the X-ray Fluorescence (XRF), and generation of measured spectral data indicative of the detected radiation. Such measurement systems are described for example in WO16157185, WO17175219, WO18051353, all assigned to the assignee of the present application, and being incorporated herein by reference.

The parameters/conditions setting different measurement schemes may include one or more of the following: parameters of the primary radiation (e.g. intensity, collimation, spot size, distribution of energy of the photons in the primary radiation, etc.); filtering parameters/conditions of the secondary radiation being detected; as well as sample's orientation with respect to the radiation source and/or detector achieved for example by rotation of the sample's support unit 14D around one or more axes (so that the counts from various sample orientations are collected in a single spectrum). Hence, it should be understood that the support unit 14D may be associated with one or more drivers for adjusting its position within a measurement plane as well as adjusting the position of the measurement plane with respect to the radiation source and/or detector. Also, the radiation source 14A may be associated with one or more drivers for adjusting/varying operation parameters of the source (e.g. current and/or voltage of a tube emitting the primary radiation; and/or filters); as well as the detector 14B may be associated with a filtering assembly for operating/varying filters at the input of the radiation detector. Additionally, geometrical characteristics of the radiation source and detector may be variable/adjustable, to improve/optimize the system performance. Such geometrical characteristics may include one or more of the following: a distance from the X-ray source to a predetermined surface region of the sample; a distance from this surface region to the detector (detection plane); angular orientation of an irradiation channel (the angle between the primary X-ray beam propagating from the X-ray source (primary beam propagation axis) and the surface of the sample); and angular orientation of a collection/detection channel (the angle between the secondary X-ray radiation coming from the sample (secondary beam axis) towards the detector and the sample's surface).

Thus, the system controller 14C is configured and operable for varying/adjusting any of the above exemplified parameters/conditions of the elements of the measurement system to define each of the K measurement schemes, and operate the measurement sessions on each sample accordingly.

During the modelled data creation, spectral measurements are performed on so-called “reference samples”, and therefore in the figure the measured data is referred to as “reference data”. The reference sample is a sample whose association with a specific group is known.

Thus, the measurement system 12 operates to apply spectral measurements to N reference samples, each sample being measured with K different measurement schemes. These N reference samples include samples relating to M groups, each g-th group (g=1, . . . , M) has predetermined different (group unique/related) characteristics. Thus, generally, the first group G₁ includes n₁ samples, second group G₂ includes n₂ samples, . . . , and M-th group G_{M} includes n_{M} samples, where

n₁+n₂+ . . . n_{M}=N

The reference data being input to (accessed by) the control system 10 (either directly from the measured system or from a storage device) includes (N×K) measured data pieces, i.e.:

(K×n₁)+(K×n₂)+ . . . (K×n_{M}).

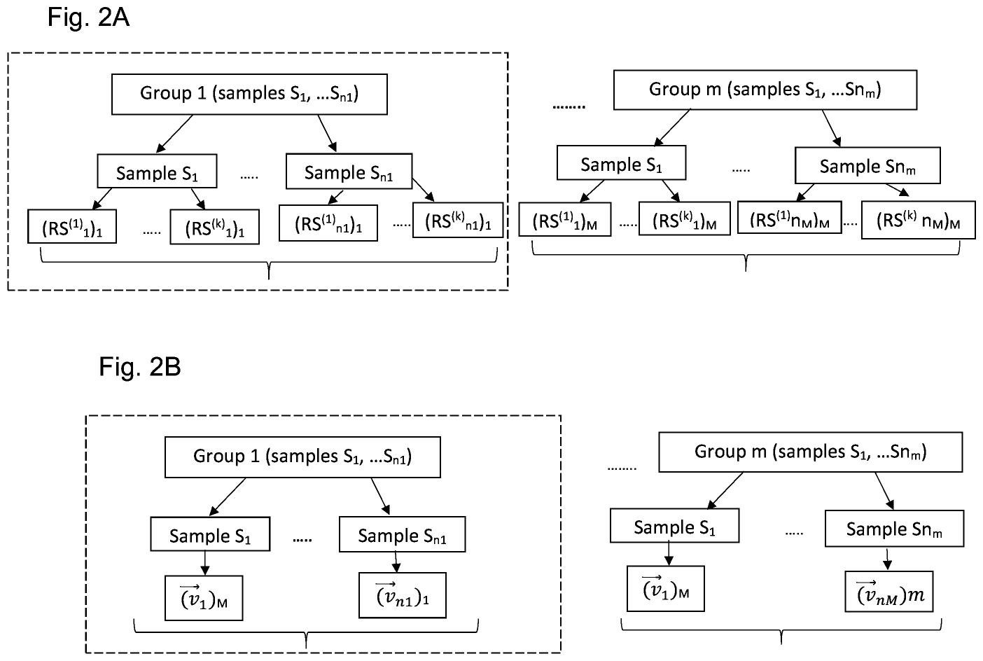

Each data piece is indicative of/corresponds to spectral response of reference sample RS. Thus, as also shown in FIG. 2A, the reference measured data include the following:

For group G₁:

(RS⁽¹⁾₁)₁, (RS⁽¹⁾₂)₁, . . . (RS⁽¹⁾n₁)₁

(RS⁽²⁾₁)₁, (RS⁽²⁾₂)₁, . . . (RS⁽²⁾n₁)₁

. . .

(RS^{(k)}₁)₁, (RS^{(k)}₂)₁, . . . (RS^{(k)}n₁)₁

For group G₂:

(RS⁽¹⁾₁)₂, (RS⁽¹⁾₂)₂, . . . (RS⁽¹⁾n₂)₂

(RS⁽²⁾₁)₂, (RS⁽²⁾₂)₂, . . . (RS⁽²⁾n₂)₂

(RS^{(K)}₁)₂, (RS^{(K)}₂)₂, . . . (RS^{(K)}n₂)₂

For group Gₘ:

(RS⁽¹⁾₁)_{M}, (RS⁽¹⁾₂)_{M}, . . . (RS⁽¹⁾n_{M})_{M}

(RS⁽²⁾₁)_{M}, (RS⁽²⁾₂)_{M}, . . . (RS⁽²⁾n_{M})_{M}

(RS^{(K)}₁)_{M}, (RS^{(K)}₂)_{M}, . . . (RS^{(K)}n_{M})_{M}

It should be understood that here the indices are as follows: (RS⁽ⁱ⁾ₙ)_{g}, wherein superscript index i corresponds to the i-th measurement scheme (i=1, . . . , K), and the subscript indices n and g correspond to the n-th sample of the g-th group. Thus for example (RS⁽²⁾₍₃₎)₄ refers to the reference spectrum of sample 3 in group 4 measured according to measurement scheme 2.

It should be understood, and will be described further below, that similar measurements are performed on an unknown sample of interest, which is to be classified, but in that case the association of the sample with the group is not known and is to be determined. Thus, in case of such unknown sample, the measured spectral data would include K spectra corresponding to different measurement schemes, being those used for the modeled data creation.

As described above, the control system 10 is configured as a computer system, which includes such main structural and functional parts/utilities as data input and output utilities 16, 18; memory 20; and data processor 22. The data processor includes a model creation module 22A, a fitting module 22B, group characterization module 22C and a weighting module 22D. The reference spectral data being received is typically stored in the memory 20 and is then used by the processor 22 to create the modeled data.

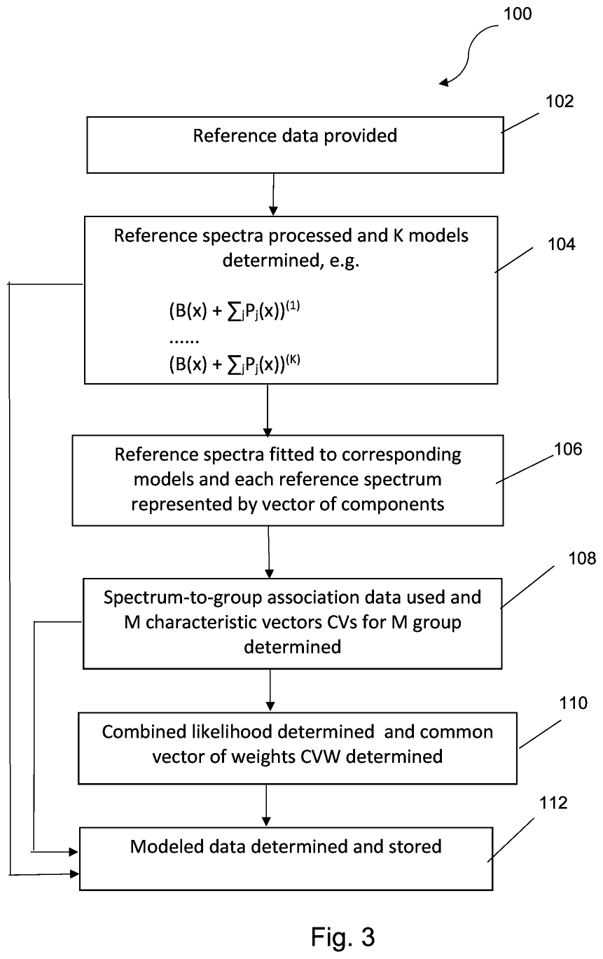

The model creation module 22A is preprogrammed to process the (N×K) measured reference spectra and determine a model for each of the K measurement schemes, i.e. determine K models describing spectral response of a sample. The model is based on a predetermined function having a spectral line shape, and relating to the respective measurement scheme. Such predetermined function of the spectral line shape may for example include Lorentzian, Gaussian or Voigt functions, whose parameters include a line position, a maximum height and width (or half-width). As will be described further below, the model may include such predetermined function of the spectral lines shape and a certain piecewise linear function. The model creation process is described more specifically further below with reference to FIG. 3.

The fitting module 22B is configured to compare each of the measured reference spectra to the model of the corresponding measurement scheme, in an iterative fitting procedure. During fitting, the model parameters are optimized via the best fit conditions, and for each reference spectrum a vector representation thereof is determined. In other words, each of the reference samples is represented by the respective vector of components. It should be understood, and will be described more specifically further below, that such vector-components representation is a combined one for all K measurement schemes; this is the sample's representation.

The group characterization module 22C operates to determine a characteristic vector of the group. To this end, the module analyzes the vectors of components of the samples based on the data indicative of correspondence of each of the reference samples to the respective one of M groups.

The weighting module 22D is configured to determine weight parameters of the vector components corresponding to maximal value of a combined likelihood for associating all the vectors of components of the reference samples with their respective groups. By this, a common vector of weights is determined (common for all the groups).

The so-determined data forms the modeled data, which includes: (i) data indicative of the K models for the respective K measurement schemes, (ii) data indicative of the characteristic vectors of the groups, and (ii) data indicative of the common vector of weights for all the groups.

Reference is now made to FIG. 3 exemplifying a flow diagram 100 of a method of the invention for generating/creating the modeled data from the measured reference data, which can be obtained as described above and includes reference spectra obtained with K measurement schemes for N reference samples relating to M groups. It should be noted that, generally, more than one spectra from the same sample and with the same measurement scheme may be obtained.

Thus, reference measured data is provided (step 102) and can be accessed either at the measurement system or separate storage device (i.e. measured data provider). Optionally, some pre-processing of the measured spectra may be carried out. This may be aimed at defining in each spectrum, region(s) of interest on which the modeling and/or classification would proceed, and/or at identifying and removing background noise and/or artifact signals from the spectra. The selected regions of interest selected may generally be affected by the measurement conditions under which the spectra are measured. Noise and artifact signals may include, for example, in cases of samples made of crystalline material, X-ray diffraction peaks due to the crystalline structure of the sample. Furthermore, in case of XRF spectra, these artifact signals may include peaks originating from materials found in the radiation source, the detector or the vicinity of the sample (not in the sample itself), as well as pileup peaks and background counts or signals origination from other processes. For the purpose of processing the spectra to remove noise, and/or artifact signals, any known suitable technique can be used, for example methods described in the above-indicated WO16157185 assigned to the assignee of the present application and incorporated herein by reference.

Thus, the reference measured spectra to be processed for the modeled data creation may be pre-processed spectra, as well as may be sample-related spectra or those of previously defined regions of interest in the samples. Such pre-processed or not reference spectral data is now processed and analyzed to create K models corresponding to the K measurement schemes used in obtaining the reference spectra (step 104). To this end, for each measurement scheme, an averaged spectrum is obtained, i.e. for the reference spectra corresponding to the same measurement scheme, averaging is performed by summing all these spectra and dividing by the number of samples. More specifically, for each i-th measurement scheme (i=1, . . . K):

$$\frac{{\sum }_{1}^{N} ( {RS}^{( i )} )}{N}$$

where Σ₁^{N}(RS⁽ⁱ⁾) is the sum spectrum corresponding to the measurement scheme:

$${\sum }_{1}^{N} ( {RS}^{( i )} ) == {( {RS}_{1}^{( i )} )}_{1} + {( {RS}_{1}^{( i )} )}_{2} … {( {RS}_{1}^{( i )} )}_{n 1} + … {( {RS}_{M}^{( i )} )}_{1} + {( {RS}_{M}^{( i )} )}_{2} … + {( {RS}_{M}^{( i )} )}_{nM}$$

$$N = {n}_{1} + {n}_{2} … + {n}_{M}$$

Thus, K such averaged spectra are determined. The averaged spectrum of each group is further processed to create the corresponding model (a so-called “mixture model”) by applying to the averaged spectrum a transformation T according to a predetermined base function BF having a spectral line shape (e.g. Gaussian) and a background function AF (e.g. a piecewise linear function or piecewise polynomial function). More specifically, for each i-th measurement scheme:

Σ₁^{N}(RS⁽ⁱ⁾)→T(BF,AF)

For example, a result of such transformation is:

T=B(x)+ΣⱼPⱼ(x),

where AF=B(x) is the background function, and BF=P(x) is the base function, which is typically in the form of multiple sub-functions (e.g. Gaussians) having different peaks in intervals, x, of the main function's domain, and index j corresponds to the j-th sub-function of the base function (having a specific Gaussian/peak).

Thus, K mixture models for the K measurement schemes, respectively, are determined (step 104):

(B(x)+ΣⱼPⱼ(x))⁽¹⁾

. . .

(B(x)+ΣⱼPⱼ(x))^{(K)}

For the purposes of the present invention, where spectral measured data is considered, the model is selected to have peak functions and a background function. The peak functions represent the peaks in the corresponding averaged spectrum, which commonly relate to materials and elements within the sample, yet may also relate to various other phenomena and processes within the sample, the vicinity of the sample (e.g. in the sample cup), the radiation source or the detector. For example, artifact peaks which may correspond to foreign materials preset at the radiation source.

In a particular non-limiting example, the measured spectra are X-ray spectra and artifact peaks may include Compton peaks, Rayleigh peaks, pileup peaks, Bremsstrahlung as well as peaks originating from other processes. The background function represents the background of the corresponding averaged spectrum.

Hence, a spectral model corresponding to the averaged spectrum measured under a particular i-th measurement condition/scheme may be in the form:

$${( B ( x ) + {\sum }_{j} {P}_{j} ( x ) )}^{( i )} ,$$

wherein B(x) is the background function, representing the background contribution to the counts or counts per second (CPS) for energy x (of the incoming photons); and the Pⱼ(x) are the peak functions representing the contribution of the peaks to the counts or CPS in photon energy x.

The peak function may be defined by a set of parameters. In an example, the peak functions are Gaussian functions G_{J}(h_{J}, σ_{J}, x̅_{J}) which are determined by such peaks' parameters (spatial features) as their height hⱼ, width σⱼ, and center position x̅ⱼ.

In a different example, the peak functions are Lorentzian functions. In an example, the background function B(x) is a spline defined by piecewise polynomial functions. In an example, the background function is an exponential polynomial.

The so-determined K models are then used to determine, for each reference spectrum, a corresponding vector of components (step 106). This is performed by fitting each reference spectrum (R⁽ⁱ⁾ₙ)_{g} (i=1, . . . K) of n-th sample of g-th group corresponding to the i-th measurement scheme, to the respective i-th model, while varying the values of the selected model parameter(s) (e.g. hⱼ, peaks' heights mostly corresponding to the peaks in the reference spectrum,) until the best fit condition is obtained. By this, a set of parameters is obtained corresponding to a reference spectrum of a particular sample of a particular measurement scheme. All K sets of parameters corresponding to a particular sample are then combined to create a single vector of parameters per reference sample. It should be understood, that this is a “combined” vector of parameters relating to/ representing the reference sample for all measurement schemes applied to the sample.

More specifically, fitting is performed by adjusting the parameters of the peaks of the model spectrum to the measured spectrum. For that purpose, one or more of the parameters of the peak functions are selected and are set so that a match between the measured reference spectrum and the model is obtained. This can be done by setting the chosen parameters so as to minimize a measure of a distance between the model (of a given measurement conditions) and the measured spectrum which is determined by the selected parameters of the peak functions and may also depend on the uncertainty in these parameters.

In the example where the selected parameters are the heights of the peak functions, the distance between the model and the measured spectrum (both corresponding to the same measurement conditions) is defined as:

$${\sum }_{r} \frac{{( {T}_{r} - {y}_{r} )}^{2}}{Δ {y}_{r}^{2}}$$

wherein: yᵣ is the measured value in the spectrum in an energy r; Tᵣ is the corresponding value of the model (transformation function) in the same energy; and Δyᵣ is the uncertainty in the measured value (depending on the type of measurements); the value of Tᵣ (model) is optimized by the best fit condition. For peak heights measured in counts 20 or counts per second, the uncertainty is \sqrt{yⱼ}.

In an example, the fitting is done iteratively, for instance by nonlinear minimization. The one or more parameters of peak function Pⱼ (included in the model T) which are set are defined as a component j in a vector of parameters corresponding to a spectrum, taken under particular measurement scheme, from a particular sample. The vector of components corresponding to a sample s is obtained by combining all parameters/components from all spectra corresponding to sample s and taken under K different measurement conditions and parameters characterizing the background to a single combined vector of components.

In an example, the peak functions representing the peaks in the models are Gaussian functions and the parameters that are set to fit the spectra of the sample to the models are the heights of the Gaussians hⱼ. Accordingly, a vector of components corresponding to the n-th sample would be of the form:

\vec{vₙ}=(hₚ . . . , h_{f} . . . , h_{q} . . . , bₗ)

wherein each of the parameter/component sets hₚ, h_{f}, and h_{q} may correspond to spectra measured under different measurement conditions, and bₗ are the background parameters.

Thus, N vectors of components, \vec{v}₁, \vec{v}₂, . . . , \vec{v}_{N}, representing N measured reference samples are obtained (step 106). This is also illustrated in FIG. 2B showing the sample's spectral data transformed into vector of components representation of the sample, in association of the reference samples to the groups.

The so-obtained sample-relating vectors of components and the known data about association of the reference samples to the groups are used to determine a characteristic vector CV for each group, i.e. M such characteristic vectors, CV₁, CV₂, . . . , CV_{M}, for the M groups (step 108). To this end, the vectors of components are processed to obtain an expression for estimating a likelihood for each sample to belong to a group (cluster of samples). This estimation may be performed as follows:

For each component j of the vector of components corresponding to the reference classified samples (belonging to g-th group), the group average (v̅_{j,g}) and the group standard deviation (σ_{j,g}) is evaluated. The average and standard deviation define a distance function, describing amount of spread of the values of the components in the vectors of components. As described above, the group' characteristic vector includes average values of the components in the vectors of components representing the reference samples of the same group. The distance function is associated with the average values and the standard deviation, which are then employed to calculate a first value for the likelihood [symbol] ₛ(g) for each classified sample s to belong to each of the groups. This can be done in a component by component manner, wherein the likelihood is defined as a product of the probabilities, pₛ(j, g, wᵢ), of each component of the vector of components (relating to sample s) to belong to g-th group:

[symbol]

ₛ(g)=Πⱼpₛ(j, g, wᵢ).

The probabilities pₛ(j, g, wᵢ) depend on the averages \vec{v}_{j,g} and the standard deviations σ_{j,g} and may depend also on non-negative weights wⱼ which initially are set to 1.

In an example the probabilities may be defined as

$${p}_{s} ( j , g , {w}_{j} ) = \frac{1}{\sqrt{2 π {σ}_{j , g}^{2}}} exp ( - {w}_{j} \frac{{( {v}_{j} - {\overline{v}}_{j , g} )}^{2}}{2 {σ}_{j , g}^{2}} )$$

Then, a common vector of weights for all the groups is determined (step 110). To this end, weight parameters wⱼ of the distance function are determined corresponding to a condition that maximizes a combined likelihood for associating all the vectors of components of the reference samples with their respective groups. This is determined based on the distance function between the vector of components of the reference sample and the characteristic vector of the group.

More specifically, optimized (final) values for the weights wⱼ are obtained by optimizing the probability, P_{corr}, for a correct classification of the classified samples into groups. The probability for a correct classification may be expressed as a product over all groups of a product over all samples in a group of the probability of the sample to belong to the group:

P_{corr}=Π_{g}Π_{s∈g}pₛ (g)

wherein the probability of sample s to belong to group g is defined as the normalized likelihood:

pₛ(g)= [symbol] ₛ(g)/Σ_{g} [symbol] ₛ(g).

In other words, the values of the weights are set so as to maximize the value of P_{corr}. The optimization process can be carried out by any nonlinear optimization method (e.g. Levenberg-Marquardt, BFGS, GRG, evolutionary methods).

As described above, the vector of weights wⱼ, together with the M characteristic vectors of the group, CV₁, CV₂, . . . , CV_{M}, and the K models corresponding to the K measurement schemes, are stored as the modeled data to be used for classifying an unknown/unclassified sample of interest.

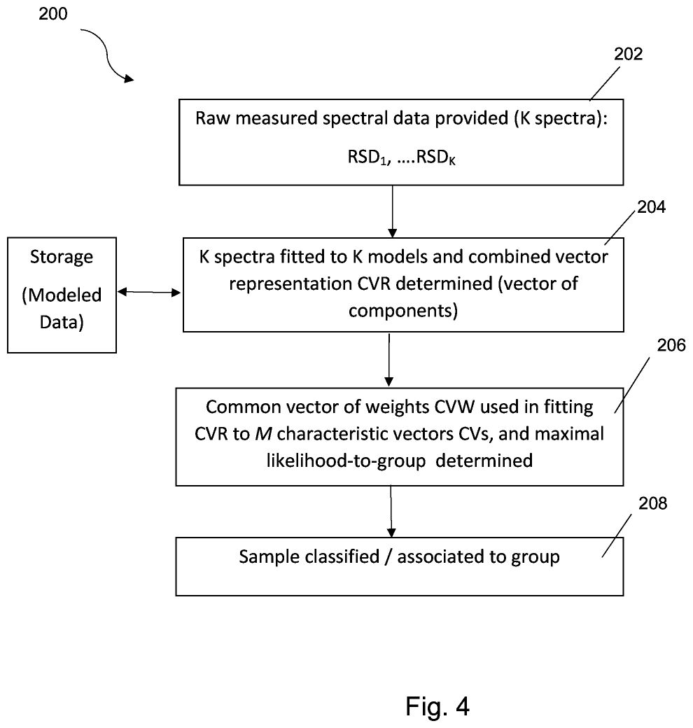

In this connection, reference is now made to FIG. 4 showing a flow diagram 200 of the exemplary method of the invention for associating the unclassified samples with a group of classified samples.

To this end, raw measured spectral data of the sample of interest is provided (step 202) corresponding to the K measurement schemes. Such measured data may be obtained as described above, using the measurement system 14. The measured data may be provided directly from the measurement system or from a separate storage device (generally, from measured data provider 12). The measured data includes K data pieces corresponding to K measured spectra of the sample of interest under the K measurement schemes, respectively: MS⁽¹⁾, MS⁽²⁾, . . . MS^{(K)}.

The measured data undergoes model-based analysis/processing using the above-described modeled data. More specifically, each i-th measured spectrum MS⁽ⁱ⁾ from the K measured spectra is fitted to the respective i-th model of the stored K models, until best fit condition is obtained, and these best fit conditions' parameters for the K measured spectra are used to create a combined vector representation CVR of the sample for all K measurement schemes—step 204. Then, this combined vector representation CVR, undergoes fitting to the groups' characteristics vectors, CV₁, CV₂, . . . , CV_{M} to determine the group-related maximal likelihood—step 206. More specifically, for the sample's combined vector representation CVR, the likelihood [symbol] ₛ(g) to belong to each of the groups (using the final values for the weights) is determined, and the group for which the likelihood is maximal is selected as the sample's related/associated group (step 208). To this end, the above-described distance function with the common vector of weights is used to determine distances of the combined vector representation of the sample to each of the characteristic vectors of the groups, and associating the sample with the group for which the determined distance is minimal.

It should be understood that the use of the models (model spectra) provides for reducing the dimensionality. Indeed, the raw data (measured spectrum) includes counts or counts per second in about 2000 spectral channels, each corresponding to an energy band (of the incoming photons). In the model, all these channels which belong to a certain peak are group together allowing to end up with a significantly smaller number of peaks (each described for example as a Gaussian function). By significantly reducing the number of parameters, reduction of resources in terms of computational power, time, etc. can be achieved. Further, the model based approach provides for reducing the noise. The noise in the counts h in a channel is \sqrt{h}, therefore the signal-to-noise ratio will be increased if the counts in a number of channels is taken.

The present invention also provides a novel technique for clustering samples, i.e. classifying samples into groups or clusters, without having prior knowledge regarding correspondence or interrelation between the samples. In this technique, there is no modeled data prepared using association of “known” reference samples to the groups/clusters. The samples are classified by studying one or more spectra of electromagnetic signals emitted from the samples. This may for example be X-ray fluorescence response of the sample to X-ray or Gamma-ray radiation.

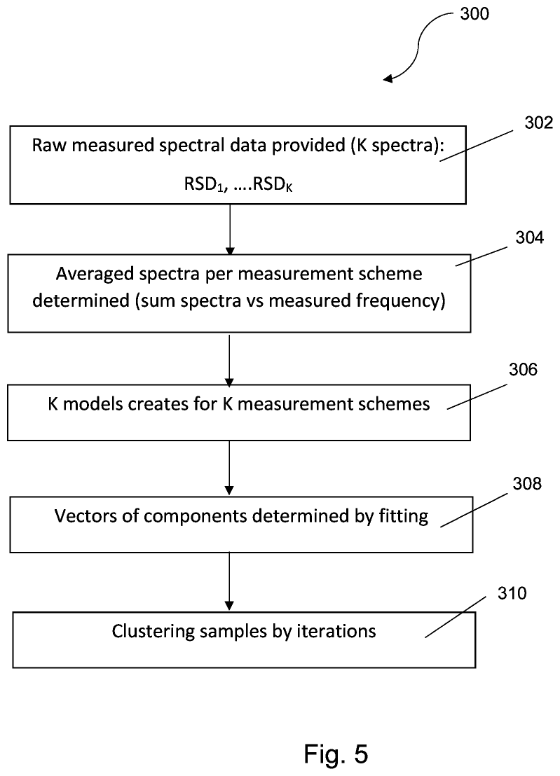

In this connection, reference is made to FIG. 5 illustrating a flow diagram 300 of the method of the invention for clustering samples. Measured data of the samples is provided including one or more spectra from each of the samples, where, similar to the above-described modeling and classifying techniques, measured data per sample includes K spectra measured under K different measurement condition/scheme (step 302). Thus, the measured data about N samples includes (N×K) spectra:

MS₁⁽¹⁾, . . . MS₁^{(K)}, . . . , MS_{N}⁽¹⁾, . . . MS_{N}^{(K)}.

Optionally, similar to the above-described techniques, the measured spectra are processed to define in each spectrum, regions of interest based on which the clustering would proceed, and identify and remove background noise and/or artifact signals from the spectra.

The measured data is processed to determine the averaged spectrum (step 304), similar to the technique described above. To this end, one or more sum spectra are determined each corresponding to the sum of counts (that is photon counts at the detector) or counts per second (CPS) of all spectra measured under the same measurement scheme vs. the measured frequency (energy) of the incoming photons arriving from the sample.

The averaged spectra are used to create the models corresponding to the K measurement schemes (step 306) in a manner described above with reference to FIGS. 1-3. Each of the measured spectrum undergoes fitting to the corresponding model (that is the spectrum model of the same measurement scheme), and for each sample, a vector of components is determined (similar to the technique described above)—step 308.

These vectors of components are used to iteratively classify the samples into groups—step 310. The classification may be performed using a clustering algorithm. In an example, clustering may be implemented by centroid based clustering algorithms. More specifically, set samples are partitioned into groups, wherein a number M of groups is determined based on some prior knowledge regarding the samples (e.g. the samples may originate from known number of sources), or randomly. The assignment of samples into the groups may be done at random. The centroid of each cluster of samples is determined by evaluating the average of each component in the vector of components associated with the samples in the cluster. The vector of the averages is defined as centroid of the cluster.

In a particular example, the clustering is carried out by K-means type algorithm wherein clustering proceeds iteratively. In each iteration the distance of each vector of parameters to each of the centroids is evaluated. The distance of a vector vⱼ from the centroid of a group v̅_{j,g} may be defined as the Euclidian distance or a normalized Euclidian distance wherein for example the distance in each component is normalized by the group standard deviation of the component

$${\sum }_{j} \frac{{( {v}_{j , g} - {\overline{v}}_{j} )}^{2}}{{σ ( {v}_{j} )}_{g}^{2}}$$

The vector of components may then be re-assigned to a different cluster if the distance to that cluster (i.e. to the centroid) is the shortest. The distance between vectors may be defined as the Euclidean distance. Additionally, other clustering methods may be used such as hierarchical clustering, density based clustering and more.

Thus, the present invention provides a novel technique for model-based analysis of measured spectral data of a sample to classify/associate the sample with a group of related/similar samples, as well as a novel technique for modeled data creation. The technique of the invention can be used in various applications dealing in clustering/ grouping the samples/objects. The data analysis system can be integral with a spectral measurement system or in a separate control system, and the data analysis process may be performed in a so-called “on-line” or off-line mode.

1. A method for model-based analysis of samples of interest, the method comprising:
providing reference data indicative of spectral measurements of a number K of measurement schemes performed on a plurality of N reference samples relating to M groups, which have predetermined different characteristics, the reference data comprising raw measured data including a plurality of (N×K) measured reference spectra, and comprising data indicative of correspondence of each of the reference samples to a respective one of said M groups;
processing said plurality of the (N×K) measured reference spectra to determine K models corresponding to said K measurement schemes, respectively, the models being based on a predetermined function having a spectral line shape, and relating to the respective measurement scheme;
fitting each of said K models with each of the N measured reference spectra corresponding to the respective measurement scheme, and creating, for each of the reference samples, a vector representation of the sample's reference spectra for said number K of measurement schemes, thereby representing each of the reference samples by the respective vector of components;
utilizing said data indicative of the correspondence of each of the samples to the respective one of said M groups, and, for each group, analyzing the vectors of components of the samples relating to the group, and determining data indicative of a characteristic vector of the group; and
determining weight parameters of a distance function that maximizes a combined likelihood for associating all the vectors of components of the reference samples with their respective groups, based on the distance function between the vector of components of the reference sample and the characteristic vector of the group, thereby providing a common vector of said weight parameters of the distance function;
storing modeled data comprising data indicative of the K models for the respective K measurement schemes, data indicative of the characteristic vectors of the group, and data indicative of the common vector of weights for the M groups, thereby enabling to classify a sample of interest to relate it to one of said M groups, by model-based analysis of raw measured spectral data of the sample of interest using said modeled data.

2. The method according to claim 1, further comprising performing said classifying of the sample of interest comprising:
based on the raw measured spectral data of the sample of interest, determining K data pieces corresponding to K measured spectra of the sample of interest under the K measurement schemes, respectively,
applying the model-based analysis to said K data pieces, said applying comprising:
using the stored K models and fitting each of said K measured spectra to the sample of interest to the respective one of the stored K models, and, based on best fit conditions for each of the K measured spectra, creating a combined vector representation of the sample of all of said K measurement schemes;
applying said distance function with said common vector of weights to determine distances of said combined vector representation of the sample to each of the characteristic vectors of the groups, and associating said sample with group for which the determined distance is minimal.

3. The method according to claim 1, wherein said number K of the measurement scheme is at least 2.

4. The method according to claim 1, wherein the model is configured as a mixture model, being based on said predetermined function of the spectral line shape and a certain piecewise polynomial function.

5. The method according to claim 1, wherein said distance function is a statistical function.

6. The method according to claim 1, wherein said characteristic vector of the group comprises average values of the components in the vectors of components representing the reference samples of the same group.

7. The method according to claim 6, wherein said distance function is associated with the average values of the components of the vectors and standard deviation, thereby describing amount of spread of the values of the components in the vectors of components.

8. The method according to claim 1, wherein said processing of the plurality of the (N×K) measured reference spectra to determine the K models comprises:
for each i-th plurality of the measured reference spectra of the N reference samples corresponding to the i-th measurement scheme, determining an average measured reference spectrum; and
applying to each i-th average measured reference spectrum a predetermined transformation according to said predetermined function having the spectral line shape, to obtain a respective i-th model corresponding to the i-th measurement scheme, thereby obtaining the K models for the K measurement schemes.

9. The method according to claim 1, wherein said predetermined function comprises a Gaussian function.

10. The method according to claim 1, wherein the sample being at least one of the following types: mineral, precision stone, diamond.

11. The method according to claim 10, wherein the predetermined different characteristics of the M groups comprise one or more of the following: one or more structural parameters of an area of sample origination, and a geographical location of an area of sample origination.

12. The method according to claim 1, wherein the measured spectral data of the sample is indicative of X-ray Fluorescence (XRF) response of the sample to X-ray or Gamma-ray radiation.

13. A data analysis system for modeling measurements on samples, the system comprising:
a measurement system configured and operable to perform spectral measurements on a plurality of N reference samples relating to M groups of predetermined different characteristics, under a number K of measurement schemes, and generate measured reference data including a plurality of (N×K) measured reference spectra in association with said M groups;
a control system configured and operable to determine, based on said measured reference data, modeled data enabling further classification of a sample of interest, the control system comprising:
a model creation module configured and operable to process said plurality of the (N×K) measured reference spectra and determine K models corresponding to said K measurement schemes, respectively, the models being based on a predetermined function having a spectral line shape, and relating to the respective measurement scheme;
a fitting module configured and operable to carry out the following: for each of said K models, fitting the model with each of the N measured reference spectra corresponding to the respective measurement scheme; and creating, for each of the reference samples, a vector representation of the sample's reference spectra for said number K of measurement schemes, thereby representing each of the reference samples by the respective vector of components;
a group characterization module configured and operable to utilize data indicative of correspondence of each of the reference samples to the respective one of said M groups, and analyze, for each group, the vectors of components of the samples relating to the group, and determining data indicative of a characteristic vector of the group; and
a weighting module configured and operable to determine weight parameters of a distance function that maximizes a combined likelihood for associating all the vectors of components of the reference samples with their respective groups, based on the distance function between the vector of components of the reference sample and the characteristic vector of the group, thereby providing a common vector of said weight parameters of the distance function; and
an output utility configured and operable to generate the modeled data to be stored, said modeled data comprising: data indicative of the K models for the respective K measurement schemes, data indicative of the characteristic vectors of the group, and data indicative of the common vector of weights for the M groups.

14. A sample classification system comprising:
a measurement system configured and operable to perform spectral measurements on samples under a number K of measurement schemes, and generate, for each of the measured samples, measured spectral data comprising K measured data pieces indicative of measured spectra corresponding to the K measurement schemes, respectively;
a control system configured and operable to communicate with the measurement system to receive the measured spectral data of a sample of interest, and configured and operable to communicate with a memory storing predetermined modeled data comprising data indicative of K models for the respective K measurement schemes based on a predetermined function having a spectral line shape, data indicative of M characteristic vectors of M predetermined group to which different samples relate, and data indicative of a common vector of weights for the M groups, said control system comprising a data processor configured and operable to apply model-based processing to the received measured spectral data of the sample of interest using said predetermined modeled data, and generate classification data indicative of relation of said specific sample of interest to one of said M predetermined groups.

15. The system according to claim 14, wherein the control system comprises:
a fitting module configured and operable to carry out the following: for each of said K measured spectra, fitting the measured spectrum to the respective model, and obtaining K best fit condition spectra; and using said K best fit condition spectra to create a combined vector representation of the sample of interest for all said K measurement schemes;
a classifier module configured and operable to utilize a predetermined distance function with said common vector of weights and determine a distances of said combined vector representation of the sample of interest to each of said M characteristic vectors of the M groups, and associate said sample of interest with a group for which the determined distance is minimal.

16. The system of claim 14, wherein said control system is further configured and operable to determine said predetermined modeled data, based on the measured spectral data corresponding to spectral reference measurements for the number K of said measurement schemes performed on a plurality of N reference samples relating to said M groups, the spectral reference data comprising a plurality of (N×K) measured reference spectra, and comprising data indicative of correspondence of each of the reference samples to a respective one of said M groups, the control system comprising:
a model creation module configured and operable to process said plurality of the (N×K) measured reference spectra and determine the K models corresponding to said K measurement schemes;
a fitting module configured and operable to carry out the following: for each of said K models, fitting the model with each of the N measured reference spectra corresponding to the respective measurement scheme; and creating, for each of the reference samples, a vector representation of the sample's reference spectra for said number K of measurement schemes, thereby representing each of the reference samples by the respective vector of components;
a group characterization module configured and operable to utilize data indicative of correspondence of each of the reference samples to the respective one of said M groups, and analyze, for each group, the vectors of components of the samples relating to the group, and determining data indicative of the characteristic vector of the group; and
a weighting module configured and operable to determine weight parameters of the predetermined distance function that maximizes a combined likelihood for associating all the vectors of components of the reference samples with their respective groups, based on the distance function between the vector of components of the reference sample and the characteristic vector of the group, thereby providing said common vector of said weight parameters of the distance function.

17. A control system for use in managing sample classification, the control system being configured and operable to communicate with a measured data provider to receive measured spectral data of a sample of interest, and configured and operable to communicate with a memory storing predetermined modeled data comprising data indicative of K models for respective K measurement schemes based on a predetermined function having a spectral line shape, data indicative of M characteristic vectors of M predetermined group to which different samples relate, and data indicative of a common vector of weights for the M groups, said control system comprising a data processor configured and operable to apply model-based processing to the received measured spectral data of the sample of interest using said predetermined modeled data, and generate classification data indicative of relation of said specific sample of interest to one of said M predetermined groups.

18. The control system according to claim 17, comprising:
a fitting module configured and operable to carry out the following: for each of said K measured spectra, fitting the measured spectrum to the respective model, and obtaining K best fit condition spectra; and using said K best fit condition spectra to create a combined vector representation of the sample of interest for all said K measurement schemes; and
a classifier module configured and operable to utilize a predetermined distance function with said common vector of weights and determine a distances of said combined vector representation of the sample of interest to each of said M characteristic vectors of the M groups, and associate said sample of interest with a group for which the determined distance is minimal.

19. The control system of claim 17, further configured and operable to determine said predetermined modeled data, based on the measured spectral data corresponding to spectral reference measurements for the number K of said measurement schemes performed on a plurality of N reference samples relating to said M groups, the spectral reference data comprising a plurality of (N×K) measured reference spectra, and comprising data indicative of correspondence of each of the reference samples to a respective one of said M groups, the control system comprising:
a model creation module configured and operable to process said plurality of the (N×K) measured reference spectra and determine the K models corresponding to said K measurement schemes;
a fitting module configured and operable to carry out the following: for each of said K models, fitting the model with each of the N measured reference spectra corresponding to the respective measurement scheme; and creating, for each of the reference samples, a vector representation of the sample's reference spectra for said number K of measurement schemes, thereby representing each of the reference samples by the respective vector of components;
a group characterization module configured and operable to utilize data indicative of correspondence of each of the reference samples to the respective one of said M groups, and analyze, for each group, the vectors of components of the samples relating to the group, and determining data indicative of the characteristic vector of the group; and
a weighting module configured and operable to determine weight parameters of the predetermined distance function that maximizes a combined likelihood for associating all the vectors of components of the reference samples with their respective groups, based on the distance function between the vector of components of the reference sample and the characteristic vector of the group, thereby providing said common vector of said weight parameters of the distance function.

20. A control system for model-based analysis of samples of interest, the control system comprising:
data input utility configured and operable to receive reference data indicative of spectral measurements of a number K of measurement schemes performed on a plurality of N reference samples relating to M groups, which have predetermined different characteristics, wherein the reference data comprises raw measured data including a plurality of (N×K) measured reference spectra, and comprises data indicative of correspondence of each of the reference samples to a respective one of said M groups;
a model creation module configured and operable to process said plurality of the (N×K) measured reference spectra to determine K models corresponding to said K measurement schemes, respectively, the models being based on a predetermined function having a spectral line shape, and relating to the respective measurement scheme;
a fitting module configured and operable to perform fitting of each of said K models with each of the N measured reference spectra corresponding to the respective measurement scheme, and creating, for each of the reference samples, a vector representation of the sample's reference spectra for said number K of measurement schemes, thereby representing each of the reference samples by the respective vector of components;
a group characterization module configured and operable to utilize said data indicative of the correspondence of each of the samples to the respective one of said M groups, and, for each group, analyze the vectors of components of the samples relating to the group, and determine data indicative of a characteristic vector of the group; and
a weighting module configured and operable to weight parameters of a distance function that maximizes a combined likelihood for associating all the vectors of components of the reference samples with their respective groups, based on the distance function between the vector of components of the reference sample and the characteristic vector of the group, and thereby provide a common vector of said weight parameters of the distance function;
a storage utility for storing modeled data comprising data indicative of the K models for the respective K measurement schemes, data indicative of the characteristic vectors of the group, and data indicative of the common vector of weights for the M groups, and
a classifier module configured and operable to classify a sample of interest to relate it to one of said M groups, by model-based analysis of the raw measured spectral data of the sample of interest using said modeled data.
